(19) United States
(12) Patent Application Publication
(10) Pub. No.: US 2022/0248670 A1
(43) Pub. Date: Aug. 11, 2022

(54) FABRIC SPRAY COMPOSITIONS

(71) Applicant: Conopco, Inc., d/b/a UNILEVER, Englewood Cliffs, NJ (US)

(72) Inventor: Christopher BOARDMAN, Liandyrnog (GB)

(73) Assignee: Conopco, Inc., d/b/a UNILEVER, Englewood Cliffs, NJ (US)

(21) Appl. No.: 17/630,021

(22) PCT Filed: Jul. 24, 2020
(86) PCT No.: PCT/EP2020/071057

§ 371 Date: Jan. 25, 2022

(30) Foreign Application Priority Data
Jul. 30, 2019 (EP) 19189218.1

(51) Int. Cl. A01N 33/12; C11D 11/00; C11D 3/50; C11D 3/48; C11D 3/30; A61L 2/22; A61L 9/14; C11D 3/00; A01N 25/06; D06M 16/00; D06M 13/00; D06M 23/06; D06M 13/463; D06M 23/02

(52) U.S. Cl. CPC A01N 33/12; A61L 2101/32; C11D 3/50; C11D 3/48; C11D 3/30; A61L 2/22; A61L 9/14; C11D 3/0068; A01N 25/06; D06M 16/00; D06M 13/005; D06M 23/06; D06M 13/463; D06M 23/02; C11D 11/0017

(57) ABSTRACT

An aqueous fabric spray composition, comprising: a. 0.0001-10 wt. % free perfume. b. 0.001 to 2 wt. % quaternary ammonium biocide.

### FIELD OF THE INVENTION

The present invention relates to fabric spray compositions comprising perfumes.

### BACKGROUND OF THE INVENTION

Consumers have increasingly busy lives, with limited time to do their laundry. On the other hand, roughly 40% of garments which go through the laundry process are not dirty and could be re-worn. This practice of over washing leads to un-necessary use of water which can be problematic particularly in water scarce regions of the world.

Various fabric re-fresh sprays have been disclosed previously. Such fabric sprays ‘refresh’ garments, reducing the number of times that a full wash is required. An important component of many sprays is perfume. One reason for this may be that perfumes elicit the idea that the clothes are clean and provides the consumer with the confidence to re-wear the garment.

However, common perfume ingredients used in fabric re-fresh sprays evaporate quickly, either in the bottle, while spraying or quickly after spraying. This leads to an unsatisfactory perfume experience for many consumers. These consumers will tend to ‘over dose’ the fabric spray, i.e. spray more than the recommended volume of the composition on to the clothes. This can be problematic, for example, excessive use of fabric sprays on many delicate fabrics leads to staining.

There is a need for improved perfume delivery onto fabrics from fabric spray compositions.

### SUMMARY OF THE INVENTION

In a first aspect of the present invention is provided an aqueous fabric spray composition, comprising:
a. 0.0001-10 wt. % free perfume.
b. 0.001 to 2 wt. % quaternary ammonium biocide comprising Benzyl C12-16 alkyl dimethyl chlorides

wherein at least 25 wt. % of the perfume composition, are perfume ingredients having a LogP of 1 to 5.

In a second aspect of the present invention is provided a method of providing perfume to fabric, wherein the spray compositions as described herein are sprayed onto a fabric surface.

In a third aspect of the present invention is a use of the spray compositions as described herein to provide perfume to fabric.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

### Aqueous Compositions

The compositions of the present invention are aqueous fabric sprays. Preferably at least 60 wt. % of the composition is water, more preferably at least 70 wt. %.

The quaternary ammonium compound biocide(s)

The spray compositions of the present invention comprise quaternary ammonium compound biocides. The quarternary ammonium compound biocides comprise Benzyl C12-16 alkyl dimethyl chlorides.

The compositions comprise 0.001 to 2 wt. % quaternary ammonium compound biocides. Preferably the compositions comprise 0.005 to 1.5 wt. % quaternary ammonium compound biocides, more preferably 0.01 to 0.8 wt. % quaternary ammonium compound biocides.

Examples of suitable quarternary ammonium compound biocides are: benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide.

Preferably the quaternary ammonium compound biocide is a cation.

The quaternary ammonium compound biocide of the present invention is not an ester linked quaternary ammonium compound, otherwise referred to as ester quats.

Preferably the quaternary ammonium compound biocide comprises a mono alkyl chain. Preferably the alkyl chain has a chain length of C8 to C18.

Exemplary biocides and biocide mixtures suitable for use in the invention are given in Table 1:

TABLE 1

| | | Active | |
|---|---|---|---|
| Material name | Supplier | level | Active substances |
| Bardac 114 | Lonza | 0.48 | ADEBAC:ADBAC:DDAC |
| BTC 2125 M80E | Stepan | 0.81 | ADEBAC:ADBAC |
| BTC 8358F | Stepan | 0.81 | ADBAC |
| BTC 1010-E | Stepan | 0.5 | DDAC |
| Sanilac 80* | Corbion | 0.8 | L-Lactic Acid |
| Tinosan HP100 | BASF | 0.3 | Diclosan |
| Citric acid | Sigma Aldrich | 1.0 | Citric Acid |
| Malonic acid | Sigma Aldrich | 1.0 | Malonic Acid |

*Purac ® Sanilac (L-Lactic Acid) is being registered as a biocide by Corbion.
ADEBAC = C12-14-alkyl [(ethylphenyl) methyl] dimethyl chlorides CAS No 85409-23-0
ADBAC = Benzyl C12-16 alkyl dimethyl chlorides CAS No. 68424-85-1
DDAC = Didecyldimethylammonium chloride CAS No 7173-51-5
L-Lactic Acid = 2-hydroxypropanoic acid (C3H6O3)

A preferred material is Bardac 114 which comprises three antimicrobial quaternary ammonium compounds (QACs) in equal measure (ADEBAC, ADBAC & DDAC) in propan-2-ol (7%) and water. The active content of Bardac 114 is: ADBAC: QAC benzyl C12-16 alkyl dimethyl, chlorides (16%) DDAC: Didecyldimethylammonium chloride (16%) ADEBAC: QAC C12-14-alkyl [(ethylphenyl) methyl] dimethyl chlorides (16%)

Most preferably the quaternary ammonium compound biocide comprises Benzyl C12-16 alkyl dimethyl chlorides.

The spray compositions of the present invention may comprise additional biocidal ingredients such as those in table 1.

For example, it has been found that combining L-Lactic Acid or racemic Lactic Acid with Bardac 114 gives good biocidal activity against P. aeruginosa at ambient temperature and short contact times and also reduces the amount of Bardac 114 needed to secure EN13697:2015 pass for spray compositions; and also avoid flammable labelling on product. We also tested racemic Lactic Acid and found it gave similar results when mixed with Bardac 114.

Diclosan, a bi-halogenated compound; 4-4′ dichloro-2-hydroxy diphenyl ether may be added to the composition in some embodiments to provide weight efficient biocidal mixtures.

### Perfumes

The compositions of the present invention comprises free perfume.

Free perfume may be present at a level selected from: less than 10%, less than 8%, and less than 5%, by weight of the spray composition. Free perfume may be present at a level selected from: more than 0.0001%, more than 0.001%, and more than 0.01%, by weight of the spray composition. Suitably free perfume is present in the spray composition in an amount selected from the range of from about 0.0001% to about 10%, preferably from about 0.001% to about 8%, more preferably from about 0.01% to about 5%, by weight of the garment refreshing composition.

Useful perfume components may include materials of both natural and synthetic origin. They include single compounds and mixtures. Specific examples of such components may be found in the current literature, e.g., in Fenaroli's Handbook of Flavor Ingredients, 1975, CRC Press; Synthetic Food Adjuncts, 1947 by M. B. Jacobs, edited by Van Nostrand; or Perfume and Flavor Chemicals by S. Arctander 1969, Montclair, N.J. (USA). These substances are well known to the person skilled in the art of perfuming, flavouring, and/or aromatizing consumer products.

A wide variety of chemicals are known for perfume use including materials such as aldehydes, ketones, esters and the like. More commonly, naturally occurring plant and animal oils and exudates comprising complex mixtures of various chemical components are known for use as perfume, and such materials can be used herein. Typical perfumes can comprise e.g. woody/earthy bases containing exotic materials such as sandalwood oil, civet and patchouli oil. The perfume also can be of a light floral fragrance e.g. rose or violet extract. Further the perfume can be formulated to provide desirable fruity odours e.g. lime, lemon or orange.

Particular examples of useful perfume components and compositions are anetole, benzaldehyde, benzyl acetate, benzyl alcohol, benzyl formate, iso-bornyl acetate, camphene, cis-citral (neral), citronellal, citronellol, citronellyl acetate, paracymene, decanal, dihydrolinalool, dihydromyrcenol, dimethyl phenyl carbinol, eucalyptol, geranial, geraniol, geranyl acetate, geranyl nitrile, cis-3-hexenyl acetate, hydroxycitronellal, d-limonene, linalool, linalool oxide, linalyl acetate, linalyl propionate, methyl anthranilate, alpha-methyl ionone, methyl nonyl acetaldehyde, methyl phenyl carbinyl acetate, laevo-menthyl acetate, menthone, iso-menthone, myrcene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, vertenex (para-tertiary-butyl cyclohexyl acetate), amyl cinnamic aldehyde, iso-amyl salicylate, beta-caryophyllene, cedrene, cinnamic alcohol, couramin, dimethyl benzyl carbinyl acetate, ethyl vanillin, eugenol, iso-eugenol, flor acetate, heliotrophine, 3-cis-hexenyl salicylate, hexyl salicylate, filial (para-tertiarybutyl-alpha-methyl hydrocinnamic aldehyde), gamma-methyl ionone, nerolidol, patchouli alcohol, phenyl hexanol, beta-selinene, trichloromethyl phenyl carbinyl acetate, triethyl citrate, vanillin, veratraldehyde, alpha-cedrene, beta-cedrene, C15H24sesquiterpenes, benzophenone, benzyl salicylate, ethylene brassylate, galaxolide (1,3,4,6,7,8-hexahydro-4,6,6,7,8,8,-hexamethyl-cyclo-penta-gamma-2-benzopyran), hexyl cinnamic aldehyde, lyral (4-(4-hydroxy-4-methyl pentyl)-3-cyclohexene-10-carboxaldehyde), methyl cedrylone, methyl dihydro jasmonate, methyl-beta-naphthyl ketone, musk ambrette, musk idanone, musk ketone, musk tibetine, musk xylol, aurantiol and phenylethyl phenyl acetate.

The perfume compositions for the present invention are preferably selected to provide fragrance for the consumer throughout their interaction with the product and during the wearing of the treated clothes. The quaternary ammonium biocide prevents evaporation of such perfumes, allowing more to be delivered to the treated clothes. The perfume composition of the present invention comprises at least 25 wt. % of the perfume composition, preferably at least 30 wt. %, more preferably at least 40 wt. % of the perfume compositions, and most preferably at least 50 wt. % of the perfume composition, perfume ingredients having a LogP of 1 to 5, preferably a Log P of 1 to 4. More preferably the perfume composition of the present invention comprises at least 25 wt. % of the perfume composition, preferably at least 30 wt. %, more preferably at least 40 wt. % of the perfume compositions, and most preferably at least 50 wt. % of the perfume composition, perfume ingredients having a LogP of 1 to 5, preferably a Log P of 1 to 4 and a boiling point of greater than 150° C.

Examples of suitable perfume ingredient include: Eucalyptol, 7-Octen-2-ol, 2,6-dimethyl-, linalool, Allyl heptanoate, Verdox, Limonene, β-Pinene, and Ligustral.

Boiling point is measured at standard pressure (760 mm Hg).

The logP of many perfume ingredients have been reported; for example, the Pomona92 database, available from Daylight Chemical Information Systems, Inc. (Daylight CIS), Irvine, Calif., contains many, along with citations to the original literature. However, the logP values are most conveniently calculated by the “CLOGP” program, also available from Daylight CIS. This program also lists experimental logP values when they are available in the Pomona92 database. The “calculated logp” (ClogP) is determined by the fragment approach of Hansch and Leo (cf., A Leo, in Comprehensive Medicinal Chemistry, Vol. 4, C. Hansch, P. G. Sammens, J. B. Taylor and C. A. Ramsden, Eds., p. 295, Pergamon Press, 1990, incorporated herein by reference). The fragment approach is based on the chemical structure of each perfume ingredient, and takes into account the numbers and types of atoms, the atom connectivity, and chemical bonding. The ClogP values, which are the most reliable and widely used estimates for this physicochemical property, are used instead of the experimental logP values in the selection of perfume ingredients herein.

It is commonplace for a plurality of perfume components to be present in a free oil perfume composition. In the compositions for use in the present invention it is envisaged that there will be three or more, preferably four or more, more preferably five or more, most preferably six or more different perfume components. An upper limit of 300 perfume components may be applied.

The free perfume of the present invention is preferably in the form of an emulsion. The particle size of the emulsion can be in the range from about 1 nm to 30 microns and preferably from about 100 nm to about 20 microns. The particle size is measured as a volume mean diameter, D[4,3], this can be measured using a Malvern Mastersizer 2000 from Malvern instruments.

Without wishing to be bound by theory, it is believed that the free perfumes of this emulsion particle size will interact with the silicone emulsion to provide improved perfume longevity on the items being sprayed.

Free oil perfume forms an emulsion in the present compositions. The emulsions may be formed outside of the composition or in situ. When formed in situ, at least one emulsifier is preferably added with the free oil perfume to stabilise the emulsion. Preferably the emulsifier is anionic or non-ionic. Examples suitable anionic emulsifiers for the free oil perfume are alkylarylsulphonates, e.g., sodium dodecylbenzene sulphonate, alkyl sulphates e.g., sodium lauryl sulphate, alkyl ether sulphates, e.g., sodium lauryl ether sulphate nEO, where n is from 1 to 20 alkylphenol ether sulphates, e.g., octylphenol ether sulphate nEO where n is from 1 to 20, and sulphosuccinates, e.g., sodium dioctylsulphosuccinate. Examples of suitable nonionic surfactants used as emulsifiers for the free oil perfume are alkylphenol ethoxylates, e.g., nonylphenol ethoxylate nEO, where n is from 1 to 50, alcohol ethoxylates, e.g., lauryl alcohol nEO, where n is from 1 to 50, ester ethoxylates, e.g., polyoxyethylene monostearate where the number of oxyethylene units is from 1 to 30 and PEG-40 hydrogenated castor oil.

### Ratio of Quaternary Ammonium Biocide to Perfume

The spray compositions of the present invention comprise a ratio of quaternary ammonium biocide:perfume of 15:1 to 1:15, more preferably 10:1 to 1:10.

### Non-Ionic Surfactants

The spray composition of the present invention preferably comprises a non-ionic surfactant. Preferably the spray comprises 0.01 to 15 w.t. % non-ionic surfactant, more preferably 0.1 to 10 w.t. % non-ionic surfactant, most preferably 0.1 to 5 w.t. % non-ionic surfactant. The correct amount of non-ionic surfactant is important can be important for achieving the desired delivery of the perfume. The spray may require sufficient surfactant to carry the surfactant, however too much surfactant will interfere with the action of the spray.

The non-ionic surfactants will preferably have an HLB value of 12 to 20, more preferably 14 to 18.

Examples of non-ionic surfactant materials include: ethoxylated materials, polyols such as polyhydric alcohols and polyol esters, alkyl polyglucosides, EO-PO block copolymers (Poloxamers). Preferably, the non-ionic surfactant is selected from ethoxylated materials.

Preferred ethoxylated materials include: fatty acid ethoxylates, fatty amine ethoxylates, fatty alcohol ethoxylates, nonylphenol ethoxylates, alkyl phenol ethoxylate, amide ethoxylates, Sorbitan(ol) ester ethoxylates, glyceride ethoxylates (castor oil or hydrogenated castor oil ethoxylates) and mixtures thereof.

More preferably, the non-ionic surfactant is selected from ethoxylated surfactants having a general formula:

R₁O(R₂O)ₓH

R₁=hydrophobic moiety.

R₂=C₂H₄ or mixture of C₂H₄ and C₃H₆ units

x=4 to 120

R1 preferably comprises 8 to 25 carbon atoms and mixtures thereof, more preferably 10 to 20 carbon atoms and mixtures thereof most preferably 12 to 18 carbon atoms and mixtures thereof. Preferably, R is selected from the group consisting of primary, secondary and branched chain saturated and/or unsaturated hydrocarbon groups comprising an alcohol, carboxy or phenolic group. Preferably R is a natural or synthetic alcohol.

R2 preferably comprises at least 50% C2H4, more preferably 75% C2H4, most preferably R2 is C2H4.

x is preferably 8 to 90 and most preferably 10 to 60.

Examples of commercially available, suitable non-ionic surfactants include: Genapol C200 ex. Clariant and Eumulgin CO40 ex. BASF.

### Malodour Ingredients

Compositions of the present invention preferably comprise anti-malodour ingredient(s). Malodour ingredients maybe in addition to traditional free perfume ingredients.

Anti-malodour agent may be present at a level selected from: less than 20%, less than 10%, and less than 5%, by weight of the garment refreshing composition. Suitably anti-malodour agent is present in the garment refreshing composition in an amount selected from the range of from about 0.01% to about 5%, preferably from about 0.1% to about 3%, more preferably from about 0.5% to about 2%, by weight of the garment refreshing composition.

Any suitable anti-malodour agent may be used. Indeed, an anti-malodour effect may be achieved by any compound or product that is effective to “trap”, “absorb” or “destroy” odour molecules to thereby separate or remove odour from the garment or act as a “malodour counteractant”.

The odour control agent may be selected from the group consisting of: uncomplexed cyclodextrin; odour blockers; reactive aldehydes; flavanoids; zeolites; activated carbon; a mixture of zinc ricinoleate or a solution thereof and a substituted monocyclic organic compound; and mixtures thereof.

As noted above, a suitable anti-malodour agent is cyclodextrin, suitably water soluble uncomplexed cyclodextrin. Suitably cyclodextrin is present at a level selected from 0.01% to 5%, 0.1% to 4%, and 0.5% to 2% by weight of the garment refreshing composition.

As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in donut-shaped rings.

Preferably, the cyclodextrins are highly water-soluble such as, alpha-cyclodextrin and/or derivatives thereof, gamma-cyclodextrin and/or derivatives thereof, derivatised beta-cyclodextrins, and/or mixtures thereof. The derivatives of cyclodextrin consist mainly of molecules wherein some of the OH groups are converted to OR groups. Cyclodextrin derivatives include, e.g., those with short chain alkyl groups such as methylated cyclodextrins, and ethylated cyclodextrins, wherein R is a methyl or an ethyl group; those with hydroxyalkyl substituted groups, such as hydroxypropyl cyclodextrins and/or hydroxyethyl cyclodextrins, wherein R is a —CH2—CH(OH)—CH3 or a —CH2CH2—OH group; branched cyclodextrins such as maltose-bonded cyclodextrins; cationic cyclodextrins such as those containing 2-hydroxy-3-(dimethylamino)propyl ether, wherein R is CH2—CH(OH)—CH2—N(CH3)2 which is cationic at low pH; quaternary ammonium, e.g., 2-hydroxy-3-(trimethylammonio)propyl ether chloride groups, wherein R is CH2—CH(OH)—CH2—N+(CH3)3Cl—; anionic cyclodextrins such as carboxymethyl cyclodextrins, cyclodextrin sulfates, and cyclodextrin succinylates; amphoteric cyclodextrins such as carboxymethyl/quaternary ammonium cyclodextrins; cyclodextrins wherein at least one glucopyranose unit has a 3-6-anhydro-cyclomalto structure, e.g., the mono-3-6-anhydrocyclodextrinse.

Highly water-soluble cyclodextrins are those having water solubility of at least about 10 g in 100 ml of water at room temperature, preferably at least about 20 g in 100 ml of water, more preferably at least about 25 g in 100 ml of water at room temperature. The availability of solubilized, uncomplexed cyclodextrins is essential for effective and efficient odour control performance. Solubilized, water-soluble cyclodextrin can exhibit more efficient odour control performance than non-water-soluble cyclodextrin when deposited onto surfaces, especially fabric.

Examples of preferred water-soluble cyclodextrin derivatives suitable for use herein are hydroxypropyl alpha-cyclodextrin, methylated alpha-cyclodextrin, methylated beta-cyclodextrin, hydroxyethyl beta-cyclodextrin, and hydroxypropyl beta-cyclodextrin. Hydroxyalkyl cyclodextrin derivatives preferably have a degree of substitution of from about 1 to about 14, more preferably from about 1.5 to about 7, wherein the total number of OR groups per cyclodextrin is defined as the degree of substitution. Methylated cyclodextrin derivatives typically have a degree of substitution of from about 1 to about 18, preferably from about 3 to about 16. A known methylated beta-cyclodextrin is heptakis-2,6-di-O-methyl-β-cyclodextrin, commonly known as DIMEB, in which each glucose unit has about 2 methyl groups with a degree of substitution of about 14. A preferred, more commercially available, methylated beta-cyclodextrin is a randomly methylated beta-cyclodextrin, commonly known as RAMEB, having different degrees of substitution, normally of about 12.6. RAMEB is more preferred than DIMEB, since DIMEB affects the surface activity of the preferred surfactants more than RAMEB. The preferred cyclodextrins are available, e.g., from Cerestar U.S.A., Inc. and Wacker Chemicals (U.S.A.), Inc.

In embodiments mixtures of cyclodextrins are used.

“Odour blockers” can be used as an anti-malodour agent to mitigate the effects of malodours. Non-limiting examples of odour blockers include 4-cyclohexyl-4-methyl-2-pentanone, 4-ethylcyclohexyl methyl ketone, 4-isopropylcyclohexyl methyl ketone, cyclohexyl methyl ketone, 3-methylcyclohexyl methyl ketone, 4-tert.-butylcyclohexyl methyl ketone, 2-methyl-4-tert.butylcyclohexyl methyl ketone, 2-methyl-5-isopropylcyclohexyl methyl ketone, 4-methylcyclohexyl isopropyl ketone, 4- methylcyclohexyl secbutyl ketone, 4-methylcyclohexyl isobutyl ketone, 2,4-dimethylcyclohexyl methyl ketone, 2,3-dimethylcyclohexyl methyl ketone, 2,2-dimethylcyclohexyl methyl ketone, 3,3-dimethylcyclohexyl methyl ketone, 4,4-dimethylcyclohexyl methyl ketone, 3,3,5-trimethylcyclohexyl methyl ketone, 2,2,6-trimethylcyclohexyl methyl ketone, 1-cyclohexyl-1-ethyl formate, 1-cyclohexyl-1-ethyl acetate, 1-cyclohexyl-1-ethyl propionate, 1-cyclohexyl-1-ethyl isobutyrate, 1-cyclohexyl-1-ethyl n-butyrate, 1-cyclohexyl-1-propyl acetate, 1-cyclohexyl-1-propyl n-butyrate, 1-cyclohexyl-2-methyl-1-propyl acetate, 2-cyclohexyl-2-propyl acetate, 2-cyclohexyl-2-propyl propionate, 2-cyc10hexyl-2-propyl isobutyrate, 2-cyc10hexyl-2-propyl nbutyrate, 5,5-dimethyl-1,3-cyclohexanedione (dimedone), 2,2-dimethyl-1,3-dioxane-4,6-dione (Meldrum's acid), spiro-[4.5]-6,10-dioxa-7,9-dioxodecane, spiro-[5.5]-1,5-dioxa-2,4-dioxoundecane, 2,2-hydroxymethyl-1,3-dioxane-4,6-dione and 1,3-cyclohexadione. Odour blockers are disclosed in more detail in U.S. Pat. Nos. 4,009,253; 4,187,251; 4,719,105; 5,441,727; and 5,861,371, incorporated herein by reference.

Reactive aldehydes can be used as anti-malodour agent to mitigate the effects of malodours. Examples of suitable reactive aldehydes include Class I aldehydes and Class II aldehydes. Examples of Class I aldehydes include anisic aldehyde, o-allyl-vanillin, benzaldehyde, cuminic aldehyde, ethylaubepin, ethyl-vanillin, heliotropin, tolyl aldehyde, and vanillin. Examples of Class II aldehydes include 3-(4′-tert.butylphenyl)propanal, 2-methyl-3-(4′-tertbutylphenyl)propanal, 2-methyl-3-(4′-isopropylphenyl)propanal, 2,2-dimethyl-3-(4-ethylphenyl)propanal, cinnamic aldehyde, a-amyl-cinnamic aldehyde, and a-hexyl-cinnamic aldehyde. These reactive aldehydes are described in more detail in U.S. Pat. No. 5,676,163. Reactive aldehydes, when used, can include a combination of at least two aldehydes, with one aldehyde being selected from acyclic aliphatic aldehydes, non-terpenic aliphatic aldehydes, non-terpenic alicyclic aldehydes, terpenic aldehydes, aliphatic aldehydes substituted by an aromatic group and bifunctional aldehydes; and the second aldehyde being selected from aldehydes possessing an unsaturation alpha to the aldehyde function conjugated with an aromatic ring, and aldehydes in which the aldehyde group is on an aromatic ring. This combination of at least two aldehydes is described in more detail in WO 00/49120. As used herein, the term “reactive aldehydes” further encompasses deodourizing materials that are the reaction products of (i) an aldehyde with an alcohol, (ii) a ketone with an alcohol, or (iii) an aldehyde with the same or different aldehydes. Such deodourizing materials can be: (a) an acetal or hemiacetal produced by means of reacting an aldehyde with a carbinol; (b) a ketal or hemiketal produced by means of reacting a ketone with a carbinol; (c) a cyclic triacetal or a mixed cyclic triacetal of at least two aldehydes, or a mixture of any of these acetals, hemiacetals, ketals, hemiketals, or cyclic triacetals. These deodorizing perfume materials are described in more detail in WO 01/07095 incorporated herein by reference.

Flavanoids can also be used as anti-malodour agent. Flavanoids are compounds based on the C6-C3-C6 flavan skeleton. Flavanoids can be found in typical essential oils. Such oils include essential oil extracted by dry distillation from needle leaf trees and grasses such as cedar, Japanese cypress, eucalyptus, Japanese red pine, dandelion, low striped bamboo and cranesbill and can contain terpenic material such as alpha-pinene, beta-pinene, myrcene, phencone and camphene. Also included are extracts from tea leaf. Descriptions of such materials can be found in JP 02284997 and JP 04030855 incorporated herein by reference.

Metallic salts can also be used as anti-malodour agents for malodour control benefits. Examples include metal salts of fatty acids. Ricinoleic acid is a preferred fatty acid. Zinc salt is a preferred metal salt. The zinc salt of ricinoleic acid is especially preferred. A commercially available product is TEGO Sorb A30 ex Evonik. Further details of suitable metallic salts is provided below.

Zeolites can be used as anti-malodour agent. A useful class of zeolites is characterized as “intermediate” silicate/aluminate zeolites. The intermediate zeolites are characterized by SiO₂/AlO₂ molar ratios of less than about 10. Preferably the molar ratio of SiO₂/AlO₂ ranges from about 2 to about 10. The intermediate zeolites can have an advantage over the “high” zeolites. The intermediate zeolites have a higher affinity for amine-type odours, they are more weight efficient for odour absorption because they have a larger surface area, and they are more moisture tolerant and retain more of their odour absorbing capacity in water than the high zeolites. A wide variety of intermediate zeolites suitable for use herein are commercially available as Valfor® CP301-68, Valfor® 300-63, Valfor® CP300-35, and Valfor® CP300-56, available from PQ Corporation, and the CBV100® series of zeolites from Conteka. Zeolite materials marketed under the trade name Abscents® and Smellrite®, available from The Union Carbide Corporation and UOP are also preferred. Such materials are preferred over the intermediate zeolites for control of sulfur-containing odours, e.g., thiols, mercaptans. Suitably the zeolite material has a particle size of less than about 10 microns and is present in the garment refreshing composition at a level of less than about 1% by weight of the garment refreshing composition.

Activated carbon is another suitable anti-malodour agent. Suitable carbon material is a known absorbent for organic molecules and/or for air purification purposes. Often, such carbon material is referred to as “activated” carbon or “activated” charcoal. Such carbon is available from commercial sources under such trade names as; Calgon—Type CPG®;Type PCB®;Type SGL®;Type CAL®;and Type OL®. Suitably the activated carbon preferably has a particle size of less than about 10 microns and is present in the garment refreshing composition at a level of less than about 1% by weight of the garment refreshing composition.

Exemplar anti-malodour agents are as follows.

ODOBAN™ is manufactured and distributed by Clean Central Corp. of Warner Robins, Ga. Its active ingredient is alkyl (C14 50%, C12 40% and C16 10%) dimethyl benzyl ammonium chloride which is an antibacterial quaternary ammonium compound. The alkyl dimethyl benzyl ammonium chloride is in a solution with water and isopropanol. Another product by Clean Control Corp. is BIOODOUR CONTROL™ which includes water, bacterial spores, alkylphenol ethoxylate and propylene glycol.

ZEOCRYSTAL FRESH AIR MIST™ is manufactured and distributed by Zeo Crystal Corp. (a/k/a American Zeolite Corporation) of Crestwood, Ill. The liquid comprises chlorites, oxygen, sodium, carbonates and citrus extract, and may comprise zeolite.

The odour control agent may comprise a “malodour counteractant” as described in US2005/0113282A1 by which is hereby incorporated by reference. In particular this malodour counteractant may comprise a mixture of zinc ricinoleate or a solution thereof and a substituted monocyclic organic compound as described at page 2, paragraph 17 whereby the substituted monocyclic organic compound is in the alternative or in combination one or more of:

1-cyclohexylethan-1-yl butyrate;

1-cyclohexylethan-1-yl acetate;

1-cyclohexylethan-1-ol;

1-(4′-methylethyl) cyclohexylethan-1-yl propionate; and

2′-hydroxy-1′-ethyl(2-phenoxy)acetate.

Synergistic combinations of malodour counteractants as disclosed at paragraphs 38-49 are suitable, for example, the compositions comprising:

(i) from about 10 to about 90 parts by weight of at least one substituted monocyclic organic compound-containing material which is:

(a) 1-cyclohexylethan-1-yl butyrate having the structure:

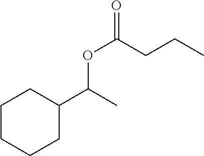

(b) 1-cyclohexylethan-1-yl acetate having the structure:

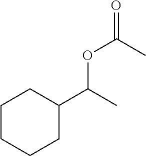

(c) 1-cyclohexylethan-1-ol having the structure:

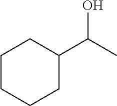

(d) 1-(4′-methylethyl)cyclohexylethan-1-yl propionate having the structure:

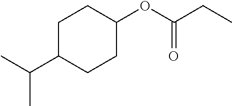

and

(e) 2′-hydroxy-1′-ethyl(2-phenoxy)acetate having the structure:

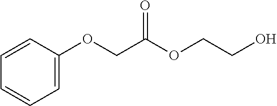 [SMILES: O=C(COc1ccccc1)OCCO]

and (ii) from about 90 to about 10 parts by weight of a zinc ricinoleate-containing composition which is zinc ricinoleate and/or solutions of zinc ricinoleate containing greater than about 30% by weight of zinc ricinoleate. Preferably, the aforementioned zinc ricinoleate-containing compositions are mixtures of about 50% by weight of zinc ricinoleate and about 50% by weight of at least one 1-hydroxy-2-ethoxyethyl ether of a More specifically, a preferred composition useful in combination with the zinc ricinoleate component is a mixture of:

(A) 1-cyclohexylethan-1-yl butyrate;

(B) 1-cyclohexylethan-1-yl acetate; and

(C) 1-(4′-methylethyl)cyclohexylethan-1-yl propionate.

More preferably, the weight ratio of components of the immediately-aforementioned zinc riconoleate-containing mixture is one where the zinc ricinoleate-containing composition: 1-cyclohexylethan-1-yl butyrate: 1-cyclohexylethan-1-yl acetate: 1-(4′-methylethyl)-cyclohexylethan-1-yl propionate is about 2:1:1:1.

Another preferred composition useful in combination with the zinc ricinoleate component or solution is a mixture of:

(A) 1-cyclohexylethan-1-yl acetate; and

(B) 1-(4′-methylethyl)cyclohexylethan-1-yl propionate.

More preferably, the weight ratio of components of the immediately-aforementioned zinc riconoleate mixture is one where the zinc ricinoleate-containing composition: 1-cyclohexylethan-1-yl acetate: 1-(4′-methylethyl)cyclohexylethan-1-yl propionate is about 3:1:1.

The anti-malodour materials of the present invention may be ‘free’ in the composition or they may be encapsulated. Suitable encapsulating material, may comprise, but are not limited to; aminoplasts, proteins, polyurethanes, polyacrylates, polymethacrylates, polysaccharides, polyamides, polyolefins, gums, silicones, lipids, modified cellulose, polyphosphate, polystyrene, polyesters or combinations thereof. Particularly preferred encapsulaing materials are aminoplasts, such as melamine formaldehyde or urea formaldehyde. The microcapsules of the present invention can be friable microcapsules and/or moisture activated microcapsules. By friable, it is meant that the perfume microcapsule will rupture when a force is exerted. By moisture activated, it is meant that the perfume is released in the presence of water.

To the extent any material described herein as an odour control agent might also be classified as another component described herein, for purposes of the present invention, such material shall be classified as an odour control agent.

### Lubricants

The spray compositions of the present invention preferably comprise lubricants. Lubricants may be silicone based lubricants or non-silicone based lubricants.

Lubricant materials may be present at a level selected from: less than 10%, less than 8%, and less than 6%, by weight of the spray composition. Lubricant materials may be present at a level selected from: more than 0.5%, more than 1%, and more than 1.5%, by weight of the spray composition. Suitably Lubricant materials are present in the spray composition in an amount selected from the range of from about 0.5% to about 10%, preferably from about 1% to about 8%, more preferably from about 1.5% to about 6%, by weight of the garment refreshing composition.

Examples of non-silicone based lubricants include fabric softening quaternary ammonium compounds, amines, fatty acid esters, clays, waxes, polyolefins, sugar polyesters, polymer latexes, synthetic and natural oils.

Preferably the lubricant is a fabric softening quaternary ammonium compounds or a silicone-based lubricant. Most preferably the lubricant is a silicone based lubricant.

For the purposes of the present invention, fabric softening quaternary ammonium compounds are so called “ester quats”. Particularly preferred materials are the ester-linked triethanolamine (TEA) quaternary ammonium compounds comprising a mixture of mono-, di- and tri-ester linked components.

Typically, TEA-based fabric softening compounds comprise a mixture of mono, di- and tri ester forms of the compound where the di-ester linked component comprises no more than 70 wt % of the fabric softening compound, preferably no more than 60 wt % e.g. no more than 55%, or even no more that 45% of the fabric softening compound and at least 10 wt % of the monoester linked component.

Preferably fabric softening quaternary ammonium compounds comprise at least one chain derived from fatty acids, more preferably at least two chains derived from a fatty acids. Generally fatty acids are defined as aliphatic monocarboxylic acids having a chain of 4 to 28 carbons. Preferably the fatty acid chains are palm or tallow fatty acids. Preferably the fatty acid chains of the QAC comprise from 10 to 50 wt. % of saturated C18 chains and from 5 to 40 wt. % of monounsaturated C18 chains by weight of total fatty acid chains. In a further preferred embodiment, the fatty acid chains of the QAC comprise from 20 to 40 wt. %, preferably from 25 to 35 wt. % of saturated C18 chains and from 10 to 35 wt. %, preferably from 15 to 30 wt. % of monounsaturated C18 chains, by weight of total fatty acid chains.

A first group of quaternary ammonium compounds (QACs) suitable for use in the present invention is represented by formula (I):

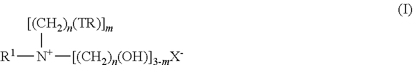

wherein each R is independently selected from a C5 to C35 alkyl or alkenyl group; R1 represents a C1 to C4 alkyl, C2 to C4 alkenyl or a C1 to C4 hydroxyalkyl group; T may be either O—CO. (i.e. an ester group bound to R via its carbon atom), or may alternatively be CO—O (i.e. an ester group bound to R via its oxygen atom); n is a number selected from 1 to 4; m is a number selected from 1, 2, or 3; and X— is an anionic counter-ion, such as a halide or alkyl sulphate, e.g. chloride or methylsulfate. Di-esters variants of formula I (i.e. m=2) are preferred and typically have mono- and tri-ester analogues associated with them. Such materials are particularly suitable for use in the present invention.

Suitable actives include soft quaternary ammonium actives such as Stepantex VT90, Rewoquat WE18 (ex-Evonik) and Tetranyl L1/90N, Tetranyl L190 SP and Tetranyl L190 S (all ex-Kao).

Also suitable are actives rich in the di-esters of triethanolammonium methylsulfate, otherwise referred to as “TEA ester quats”.

Commercial examples include Preapagen™ TQL (ex-Clariant), and Tetranyl™ AHT-1 (ex-Kao), (both di-[hardened tallow ester] of triethanolammonium methylsulfate), AT-1 (di-[tallow ester] of triethanolammonium methylsulfate), and L5/90 (di-[palm ester] of triethanolammonium methylsulfate), (both ex-Kao), and Rewoquat™ WE15 (a di-ester of triethanolammonium methylsulfate having fatty acyl residues deriving from C10-C20 and C16-C18 unsaturated fatty acids) (ex-Evonik).

A second group of QACs suitable for use in the invention is represented by formula (II):

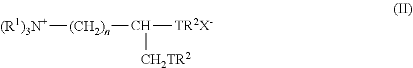

wherein each R1 group is independently selected from C1 to C4 alkyl, hydroxyalkyl or C2 to C4 alkenyl groups; and wherein each R2 group is independently selected from C8 to C28 alkyl or alkenyl groups; and wherein n, T, and X— are as defined above.

Preferred materials of this second group include 1,2 bis[tallowoyloxy]-3-trimethylammonium propane chloride, 1,2 bis[hardened tallowoyloxy]-3-trimethylammonium propane chloride, 1,2-bis[oleoyloxy]-3-trimethylammonium propane chloride, and 1,2 bis[stearoyloxy]-3-trimethylammonium propane chloride. Such materials are described in U.S. Pat. No. 4, 137,180 (Lever Brothers). Preferably, these materials also comprise an amount of the corresponding mono-ester.

A third group of QACs suitable for use in the invention is represented by formula (III):

(R¹)₂—-N⁺—[(CH₂)ₙ-T-R²]₂X⁻ (III)

wherein each R1 group is independently selected from C1 to C4 alkyl, or C2 to C4 alkenyl groups; and wherein each R2 group is independently selected from C8 to C28 alkyl or alkenyl groups; and n, T, and X— are as defined above. Preferred materials of this third group include bis(2-tallowoyloxyethyl)dimethyl ammonium chloride, partially hardened and hardened versions thereof.

A particular example of the fourth group of QACs is represented the by the formula:

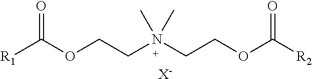

A forth group of QACs suitable for use in the invention are represented by formula (V)

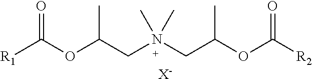

R1 and R2 are independently selected from 010 to C22 alkyl or alkenyl groups, preferably C14 to C20 alkyl or alkenyl groups. X— is as defined above.

The iodine value of the quaternary ammonium fabric conditioning material is preferably from 0 to 80, more preferably from 0 to 60, and most preferably from 0 to 45. The iodine value may be chosen as appropriate. Essentially saturated material having an iodine value of from 0 to 5, preferably from 0 to 1 may be used in the compositions of the invention. Such materials are known as “hardened” quaternary ammonium compounds.

A further preferred range of iodine values is from 20 to 60, preferably 25 to 50, more preferably from 30 to 45. A material of this type is a “soft” triethanolamine quaternary ammonium compound, preferably triethanolamine di-alkylester methylsulfate. Such ester-linked triethanolamine quaternary ammonium compounds comprise unsaturated fatty chains.

If there is a mixture of quarternary ammonium materials present in the composition, the iodine value, referred to above, represents the mean iodine value of the parent fatty acyl compounds or fatty acids of all of the quarternary amonium materials present. Likewise, if there is any saturated quaternary ammonium materials present in the composition, the iodine value represents the mean iodine value of the parent acyl compounds of fatty acids of all of the quaternary ammonium materials present.

Iodine value as used in the context of the present invention refers to, the fatty acid used to produce the QAC, the measurement of the degree of unsaturation present in a material by a method of nmr spectroscopy as described in Anal. Chem., 34, 1136 (1962) Johnson and Shoolery.

A further type of softening compound may be a non-ester quaternary ammonium material represented by formula (VI):

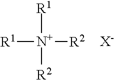

wherein each R1 group is independently selected from C1 to C4 alkyl, hydroxyalkyl or C2 to C4 alkenyl groups; R2 group is independently selected from C8 to C28 alkyl or alkenyl groups, and X— is as defined above.

Silicones and their chemistry are described in, for example in The Encyclopaedia of Polymer Science, volume 11, p765.

Silicones suitable for the present invention are fabric softening silicones. Non-limiting examples of such silicones include:
Non-functionalised silicones such as polydimethylsiloxane (PDMS),
Functionalised silicones such as alkyl (or alkoxy) functionalised, alkylene oxide functionalised, amino functionalised, phenyl functionalised, hydroxy functionalised, polyether functionalised, acrylate functionalised, siliconhydride functionalised, carboxy functionalised, phosphate functionalised, sulphate functionalised, phosphonate functionalised, sulphonic functionalised, betaine functionalised, quarternized nitrogen functionalised and mixtures thereof.
Copolymers, graft co-polymers and block co-polymers with one or more different types of functional groups such as alkyl, alkylene oxide, amino, phenyl, hydroxy, polyether, acrylate, siliconhydride, carboxy, phosphate, sulphonic, phosphonate, betaine, quarternized nitrogen and mixtures thereof.

Suitable non-functionalised silicones have the general formula:

R₁—Si(R₃)₂—O—[—Si(R₃)₂—O—]ₓ—Si(R₃)₂—R₂

R₁=hydrogen, methyl, methoxy, ethoxy, hydroxy, propoxy, and aryloxy group.

R₂=hydrogen, methyl, methoxy, ethoxy, hydroxy, propoxy, and aryloxy group.

R₃=alkyl, aryl, hydroxy, or hydroxyalkyl group, and mixtures thereof

Suitable functionalised silicones may be anionic, cationic, or non-ionic functionalised silicones.

The functional group(s) on the functionalised silicones are preferably located in pendent positions on the silicone i.e. the composition comprises functionalised silicones wherein the functional group(s) are located in a position other than at the end of the silicone chain. The terms ‘terminal position’ and ‘at the end of the silicone chain’ are used to indicate the terminus of the silicone chain.

When the silicones are linear in nature, there are two ends to the silicone chain. In this case the anionic silicone preferably contains no functional groups located on a terminal position of the silicone.

When the silicones are branched in nature, the terminal position is deemed to be the two ends of the longest linear silicone chain. Preferably no functional group(s) are located on the terminus of the longest linear silicone chain.

Preferred functionalised silicones are those that comprise the anionic group at a mid-chain position on the silicone. Preferably the functional group(s) of the functionalised silicone are located at least five Si atoms from a terminal position on the silicone. Preferably the functional groups are distributed randomly along the silicone chain.

For best performance, it is preferred that the silicone is selected from: carboxy functionalised silicone; anionic functionalised silicone; non-functionalised silicone; and mixtures thereof. More preferably, the silicone is selected from: carboxy functionalised silicone; amino functionalised silicone; polydimethylsiloxane (PDMS) and mixtures thereof. Preferred features of each of these materials are outlined herein. Most preferably the silicone is selected from amino functionalised silicones; polydimethylsiloxane (PDMS) and mixtures thereof.

A carboxy functionalised silicone may be present as a carboxylic acid or an carbonate anion and preferably has a carboxy group content of at least 1 mol % by weight of the silicone polymer, preferably at least 2 mol %. Preferably the carboxy group(s) are located in a pendent position, more preferably located at least five Si atoms from a terminal position on the silicone. Preferably the caboxy groups are distributed randomly along the silicone chain. Examples of suitable carboxy functional silicones include FC 220 ex. Wacker Chemie and X22-3701E ex. Shin Etsu.

An amino functionalised silicone means a silicone containing at least one primary, secondary or tertiary amine group, or a quaternary ammonium group. The primary, secondary, tertiary and/or quaternary amine groups are preferably located in a pendent position, more preferably located at least five Si atoms from a terminal position on the silicone. Aminosilicones suitable for use in the invention will preferably have an amine content of the composition of 0.001 to 3 meq/g, more preferably 0.01 to 2.5 meq/g, most preferably 0.05 to 1.5 meq/g, which is measured as the consumption of 1 N hydrochloric acid in ml/g by the composition on titration to the neutral point. Preferably the amino groups are distributed randomly along the silicone chain. Examples of suitable amino functional silicones include FC222 ex. Wacker Chemie and EC218 ex. Wacker Chemie.

A polydimethylsiloxane (PDMS) polymer has the general formula:

R₁—Si(CH₃)₂—O—[—Si(CH₃)₂—O—]ₓ—Si(CH₃)₂—R₂

R₁=hydrogen, methyl, methoxy, ethoxy, hydroxy, propoxy, and aryloxy group.

R₂=hydrogen, methyl, methoxy, ethoxy, hydroxy, propoxy, and aryloxy group.

A suitable example of a PDMS polymer is E22 ex. Wacker Chemie.

The molecular weight of the silicone polymer is preferably from 1,000 to 500,000, more preferably from 2,000 to 250,000 even more preferably from 5,000 to 200,000.

The silicone of the present invention is in the form of an emulsion. Silicones are preferably emulsified prior to addition to the present compositions. Silicone compositions are generally supplied from manufacturers in the form of emulsions.

The average particle size of the emulsion is in the range from about 1 nm to 150 nm, preferably 1 nm to 100 nm. This may be referred to as a micro emulsion. The particle size is measured as a volume mean diameter, D[4,3], this can be measured using a Malvern Mastersizer 2000 from Malvern instruments.

The particle size of the silicone emulsion will provides shape rejuvenation for the fabric.

### Setting Polymers

The fabric spray of the present invention may preferably further comprise one or more setting polymers. “setting polymer” means any polymer which refers to polymer having properties of film-formation, adhesion, or coating deposited on a surface on which the polymer is applied.

The setting polymer may be present at a level selected from: less than 10%, less than 7.5%, and less than 5%, by weight of the spray composition. The setting polymer may be present at a level selected from: more than 0.5%, more than 1%, and more than 1.5%, by weight of the spray composition. Suitably the setting polymer is present in the spray composition in an amount selected from the range of from about 0.5% to about 10%, preferably from about 1% to about 7.5%, more preferably from about 1.5% to about 5%, by weight of the fabric spray composition.

The molecular weight of the setting polymer is preferably from 1,000 to 500,000, more preferably from 2,000 to 250,000 even more preferably from 5,000 to 200,000.

The setting polymer according to the present invention may be any water-soluble or water dispersible polymer. Preferably the polymer is a film-forming polymer or mixture of such polymers. This includes homopolymers or copolymers of natural or synthetic origin having functionality rendering the polymers water-soluble such as hydroxyl, amine, amide or carboxyl groups. The setting polymers may be cationic, anionic, non-ionic or amphoteric. The polymers make be a single species of polymer or a mixture thereof. Preferably the setting polymer is selected from: anionic polymers, non-ionic polymers, amphoteric polymers and mixtures thereof. For all polymers herein described it is intended to cover both the acids and salts thereof.

Suitable cationic setting polymers are preferably selected from the group consisting of: quaternized acrylates or methacrylates; quaternary homopolymers or copolymers of vinylimidazole; homopolymers or copolymers comprising a quaternary dimethdiallyl ammonium chloride; cationic polysaccharides; cationic cellulose derivatives; chitosans and derivatives thereof; and mixtures thereof.

Quaternized acrylates or methacrylates are preferably selected from: copolymers comprising: a) at least one of: quaternized dialkylaminoalkyl acrylamides (e.g. Quaternized dimethyl amino propyl methacrylamide); or quaternized dialkylaminoalkyl acrylates (e.g. quaternized dimethyl aminoethyl methacrylate) and b) one or more monomers selected from the group consisting of: vinyllactams such as vinylpyrrolidone or vinylcaprolactam; acrylamides, methacrylamides which may or may not be substituted on the nitrogen by lower alkyl groups (C1-C4) (e.g. N-tertbutylacrylamide); esters of acrylic acid and/or methacrylic acid (e.g. C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g. poly(ethyleneglycol)acrylate); hydroxyesters acrylate (e.g. hydroxyethyl methacrylate); hydroxyalkylated acrylamide;

amino alkylated acrylamide (e.g. dimethyl amino propyl methacrylamide); alkylacrylamine (e.g. tert-butylamino-ethyl methacrylate, dimethyl aminoethyl methacrylate); alkylether acrylate (e.g. 2-ethoxyethyl acrylate); monoethylenic monomer such as ethylene, styrene; vinyl esters (e.g. vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; and allyl esters or methallyl esters; and mixtures thereof. The counter ion can be either a methosulfate anion or a halide such as chloride or bromide.

Quaternary homopolymers or copolymers of vinylimidazole are preferably selected from: copolymers comprising a) a quaternized vinylimizazole and b) one or more other monomers. The other monomer may be selected from the group consisting of: vinyllactams such as vinylpyrrolidone or vinylcaprolactam such as vinylpyrrolidone/quaternized vinylimidazole (PQ-16) such as that sold as Luviquat FC-550 by BASF; acrylamides, methacrylamides which may or may not be substituted on the nitrogen by lower alkyl groups (C1-C4) (e.g. N-tertbutylacrylamide); esters of acrylic acid and/or methacrylic acid (e.g. C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g. poly(ethyleneglycol)acrylate); hydroxyesters acrylate (e.g. hydroxyethyl methacrylate); hydroxyalkylated acrylamide; amino alkylated acrylamide (e.g. dimethyl amino propyl methacrylamide); alkylacrylamine (e.g. tert-butylamino-ethyl methacrylate, dimethyl aminoethyl methacrylate); alkylether acrylate (e.g. 2-ethoxyethyl acrylate); monoethylenic monomer such as ethylene, styrene; vinyl esters (e.g. vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters; and mixtures thereof. The counter ion can be either a methosulfate anion or a halide such as chloride or bromide.

Dimethdiallyl ammonium chlorides are preferably selected from: a homopolymer or copolymer comprising a quaternary dimethdiallyl ammonium chloride and another monomer. The other monomer may be selected from the group consisting of: acrylamides, methacrylamides which may or may not be substituted on the nitrogen by lower alkyl groups (C1-C4) (e.g. N-tertbutylacrylamide); vinyllactams such as vinylpyrrolidone or vinylcaprolactam; esters of acrylic acid and/or methacrylic acid (e.g. C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g. poly(ethyleneglycol)acrylate); hydroxyesters acrylate (e.g. hydroxyethyl methacrylate); hydroxyalkylated acrylamide; amino alkylated acrylamide (e.g. dimethyl amino propyl methacrylamide); alkylacrylamine (e.g. tert-butylamino-ethyl methacrylate, dimethyl aminoethyl methacrylate); alkylether acrylate (e.g. 2-ethoxyethyl acrylate); monoethylenic monomer such as ethylene, styrene; vinyl esters (e.g. vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters; and mixtures thereof. The counter ion can be either a methosulfate anion or a halide such as chloride or bromide.

Cationic polysaccharides are preferably selected from: cationic celluloses; cationic starches; cationic glycogens; cationic chitins; cationic guar gums such as those containing trialkylammonium cationic groups, for example, such as guar hydroxypropyltrimonium chloride, which is available as N-Hance 3269 from Ashland; and mixtures thereof.

Cationic cellulose derivatives are preferably selected from: a copolymers of cellulose derivatives such as hydroxyalkylcelluloses (e.g. hydroxymethyl-, hydroxyethyl- or hydroxypropylcelluloses) grafted with a water-soluble monomer comprising a quaternary ammonium (e.g. glycidytrimethyl ammonium, methacryloyloxyethyltrimethylammonium, or a methacrylamidopropyltrimethylammonium, or dimethyldiallylammonium salt) and mixtures thereof. For example, such as hydroxyethylcellulose dimethyldiallyammonium chloride [PQ4] sold as Celquat L200 by Akzo Nobel, or such as Quaternized hydroxyethylcellulose [PQ10] sold as UCARE JR125 by Dow Personal Care.

Chitosans and derivatives thereof are preferably selected from: chitosan and salts of chitosans. The salts can be chitosan acetate, lactate, glutamate, gluconate or pyrrolidinecarboxylate preferably with a degree of hydrolysis of at least 80%; and mixtures thereof. A suitable chitosan includes Hydagen HCMF by Cognis.

Suitable anionic setting polymers may be selected from polymers comprising groups derived from carboxylic or sulfonic acids. Copolymers containing acid units are generally used in their partially or totally neutralized form, more preferably totally neutralized. Suitable anionic setting polymer may comprise: (a) at least one monomer derived from a carboxylic acid such as acrylic acid, or methacrylic acid or crotonic acid or their salts, or C4-C8 monounsaturated polycarboxylic acids or anhydrides (e.g. maleic, furamic, itaconic acids and their anhydrides), or sulfonic acid such as vinylsulfonic, styrenesulfonic, naphthalenesulfonic, acrylalkyl sulfonic, acrylamidoalkyls ulfonic acid or their salts and (b) one or more monomers selected from the group consisting of: esters of acrylic acid and/or methacrylic acid (e.g. C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g. poly(ethyleneglycol)acrylate); hydroxyesters acrylate (e.g. hydroxyethyl methacrylate); acrylamides, methacrylamides which may or may not be substituted on the nitrogen by lower alkyl groups (C1-C4); N-alkylated acrylamide (e.g. N-tertbutylacrylamide); hydroxyalkylated acrylamide; amino alkylated acrylamide (e.g. dimethyl amino propyl methacrylamide); alkylacrylamine (e.g. tert-butylamino-ethyl methacrylate, dimethyl aminoethyl methacrylate); alkylether acrylate (e.g. 2-ethoxyethyl acrylate); monoethylenic monomer such as ethylene, styrene; vinyl esters (e.g. vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters; vinyllactams such as vinylpyrrolidone or vinylcapro lactam; alkyl maleimide, hydroxyalkyl maleimide (e.g. Ethyl/Ethanol Maleimide); and mixtures thereof. When present the anhydride functions of these polymers can optionally be monoesterified or monoamidated.

Alternatively, the anionic setting polymer may be selected from a water-soluble polyurethane. The polyurethane is preferably dispersed in water. Suitable polyurethanes include those such as adipic acid, 1-6 hexandiol, neopentyl glycol, isophorone diisocyanate, isophorone diamine, N-(2-aminoethyl)-3-aminoethanesulphonic acid, sodium salt (also known as Polyurethane-48) such as that sold as Baycusan C1008 by Bayer; and such as isophorone diisocyanate, dimethylol propionic acid, 4,4-isopropylidenediphenol/propylene oxide/ethylene oxide (also known as Polyurethene-14) such as that sold as a mixture under the name of DynamX H20 by Akzo Nobel; and mixtures thereof.

Alternatively, the anionic setting polymer may be selected from anionic polysaccharides. Anionic polysaccharides are preferably selected from: anionic celluloses, derivatives of anionic celluloses; anionic starches; anionic glycogens; anionic chitins; anioinc guar gums; and mixtures thereof.

Preferred anionic setting polymers may be selected from: copolymers derived from acrylic acid such as the acrylic acid/ethylacrylate/N-tert-butylacrylamide terpolymer such as that sold as Ultrahold 8 by BASF; Octylacrylamide/Acrylates/Butylaminoethyl/Methacrylate Copolymer such as that sold as Amphomer by Akzo Nobel, preferably Acrylates/Octylacrylamide Copolymer sold as Amphomer 4961; methacrylic acid/ester acrylate/ester methacrylate such as that sold as Balance CR by Akzo Nobel; a copolymer of butyl acrylate/methacrylic acid/methylmethacrylate; Octylacrylamide/Acrylates/Butylaminoethyl Methacrylate Copolymer such as that sold as Balance 47 by Akzo Nobel; methacrylic acid/hydroxyethylmethacrylate/various acrylate esters such as that known as Acudyne 1000 sold by Dow Chemical; acrylates/hydroxyethylmethacrylate such as that sold as Acudyne 180 by Dow Chemical; methacrylic acid/hydroxyethylmethacrylate/various acrylate esters such as that sold as Acudyne DHR by Dow Chemical; n-butyl methacrylate/methacrylic acid/ethyl acrylate copolymer such as that sold as Tilamar Fix A-1000 by DSM; copolymers derived from crotonic acid, such as vinyl acetate/vinyl tertbutylbenzoate/crotonic acid terpolymers and the crotonic acid/vinyl acetate/vinyl neododecanoate terpolymers such as that sold as Resin 282930 by Akzo Nobel. Preferred setting polymers derived from sulfonic acid include: sodium polystyrene sulfonate sold as Flexan 130 by Ashland; sulfopolyester (also known as Polyester-5) such as that sold as Eastman AQ 48 by Eastman; sulfopolyester (also known as Polyester-5) such as that sold as Eastman AQ S38 by Eastman; sulfopolyester (also known as Polyester-5) such as that sold as Eastman AQ 55 by Eastman; and mixtures thereof.

More preferably the anionic polymer is selected from: copolymers derived from acrylic acid such as the acrylic acid/ethylacrylate/N-tert-butylacrylamide terpolymers; Octylacrylamide/Acrylates/Butylaminoethyl/Methacrylate Copolymers; methacrylic acid/ester acrylate/ester methacrylates; Octylacrylamide/Acrylates/Butylaminoethyl Methacrylate Copolymer; methacrylic acid/hydroxyethylmethacrylate/various acrylate esters; acrylates/hydroxyethylmethacrylate; methacrylic acid/hydroxyethylmethacrylate/various acrylate esters; n-butyl methacrylate/methacrylic acid/ethyl acrylate copolymers; copolymers derived from crotonic acid, such as vinyl acetate/vinyl tertbutylbenzoate/crotonic acid terpolymers; a copolymer of butyl acrylate/methacrylic acid/methylmethacrylate; the crotonic acid/vinyl acetate/vinyl neododecanoate terpolymers; isophorone diisocyanate, dimethylol propionic acid, 4,4-isopropylidenediphenol/propylene oxide/ethylene oxide (also known as Polyurethene-14) such as that sold as a mixture under the name of DynamX H20 by Akzo Nobeland mixtures thereof.

Non-ionic setting polymers may be natural, synthetic or mixtures thereof.

Synthetic non-ionic setting polymers are selected from: homopolymers and copolymers comprising: (a) at least one of the following main monomers: vinylpyrrolidone; vinyl esters grafted onto a polyalkylene glycol such as polyethylene glycol; acrylate esters grafted onto a polyalkylene glycol such as polyethylene glycol or acrylamide and (b) one or more other monomers such as vinyl esters (e.g. vinyl acetate or vinyl propionate, vinyl tert-butyl-benzoate); alkylacrylamine (e.g. tert-butylamino-ethyl methacrylate, dimethyl aminoethyl methacrylate); vinylcaprolactam; hydroxyalkylated acrylamide; amino alkylated acrylamide (e.g. dimethyl amino propyl methacrylamide); vinyl ether; alkyl maleimide, hydroxyalkyl maleimide (e.g. Ethyl/Ethanol Maleimide); and mixtures thereof.

Suitable natural non-ionic setting polymers are water-soluble. Preferred natural non-ionic polymers are selected from: non-ionic polysaccharides including: non-ionic cellulose, non-ionic starches, non-ionic glycogens, non-ionic chitins and non-ioinc guar gums; cellulose derivative, such as hydroxyalkylcelluloses (e.g. hydroxymethyl-, hydroxyethyl- or hydroxypropylcelluloses) and mixtures thereof.

The non-ionic setting polymers are preferably selected from vinylpyrrolidone/vinyl acetate copolymers and such as vinylpyrrolidone homopolymer.

Amphoteric setting polymers may be natural, synthetic or a mixture thereof. Suitable synthetic amphoteric setting polymers include those comprising: an acid and a base like monomer; a carboxybetaine or sulfobetaine zwitterionic monomer; and an alkylamine oxide acrylate monomer.

Suitable amphoteric setting polymers comprising acid and base monmers are preferably selected from: (a) at least one monomer containing a basic nitrogen atom such as a quaternized dialkylaminoalkyl acrylamide (e.g. Quaternized dimethyl amino propyl methacrylamide) or a quaternized dialkylaminoalkyl acrylate (e.g. quaternized dimethyl aminoethyl methacrylate) and (b) at least one acid monomer comprising one or more carboxylic or sulfonic groups such as acrylic acid, or methacrylic acid or crotonic acid or their salts, or C4-C8 monounsaturated polycarboxylic acids or anhydrides (e.g. maleic, furamic, itaconic acids and their anhydrides) and (c) one or more monomers selected from acrylamides, methacrylamides which may or may not be substituted on the nitrogen by lower alkyl groups (C1-C4) (e.g. N-tertbutylacrylamide); vinyllactams such as vinylpyrrolidone or vinylcapro lactam; esters of acrylic acid and/or methacrylic acid (e.g. C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g. poly(ethyleneglycol)acrylate); hydroxyesters acrylate (e.g. hydroxyethyl methacrylate); hydroxyalkylated acrylamide; amino alkylated acrylamide (e.g. dimethyl amino propyl methacrylamide); alkylacrylamine (e.g. tert-butylamino-ethyl methacrylate, dimethyl aminoethyl methacrylate); alkylether acrylate (e.g. 2-ethoxyethyl acrylate); monoethylenic monomer such as ethylene, styrene; vinyl esters (e.g. vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters; and mixtures thereof.

Suitable amphoteric setting polymers comprising carboxybetaine or sulfobetaine zwitterionic monomer are preferably selected from: carboxybetaine methacrylate and sulfobetaine methacrylate. For example: (a) at least one carboxybetaine or sulfobetaine zwitterioni monomer such as carboxybetaine methacrylate and sulfobetaine methacrylate; and (b) a monomer selected from the group consisting of: acrylamides, methacrylamides which may or may not be substituted on the nitrogen by lower alkyl groups (C1-C4) (e.g. N-tertbutylacrylamide); vinyllactams such as vinylpyrrolidone or vinylcapro lactam; esters of acrylic acid and/or methacrylic acid (e.g. C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g. poly(ethyleneglycol)acrylate); hydroxyesters acrylate (e.g. hydroxyethyl methacrylate); hydroxyalkylated acrylamide; amino alkylated acrylamide (e.g. dimethyl amino propyl methacrylamide); alkylacrylamine (e.g. tert-butylamino-ethyl methacrylate, dimethyl aminoethyl methacrylate); alkylether acrylate (e.g. 2-ethoxyethyl acrylate); monoethylenic monomer such as ethylene, styrene; vinyl esters (e.g. vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters; and mixtures thereof.

Suitable amphoteric setting polymers comprising alkylamine oxide acrylate are perferably selected from: (a) an ethylamine oxide methacrylate; and (b) a monomer selected from the group consisting of: acrylamides, methacrylamides which may or may not be substituted on the nitrogen by lower alkyl groups (C1-C4) (e.g. N-tertbutylacrylamide); vinyllactams such as vinylpyrrolidone or vinylcapro lactam; esters of acrylic acid and/or methacrylic acid (e.g. C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g. poly(ethyleneglycol)acrylate); hydroxyesters acrylate (e.g. hydroxyethyl methacrylate); hydroxyalkylated acrylamide; amino alkylated acrylamide (e.g. dimethyl amino propyl methacrylamide); alkylacrylamine (e.g. tert-butylamino-ethyl methacrylate, dimethyl aminoethyl methacrylate); alkylether acrylate (e.g. 2-ethoxyethyl acrylate); monoethylenic monomer such as ethylene, styrene; vinyl esters (e.g. vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters. An example of such an amphoteric setting polymer is acrylates/ethylamine oxide methacrylate sold as Diaformer Z 731 N by Clariant; and mixtures thereof.

Preferably the setting polymer is selected from acrylate polymers, co-polymers comprising acrylate monomers, starches, celluloses, derivatives of cellulose and mixtures thereof.

Most preferably the setting polymer is selected from the group consisting of: acrylates and copolymers of two or more acrylate monomers such as:(meth)acrylic acid or one of their simple esters; octylacrylamide/acrylate/butylaminoethyl methacrylate copolymers; acrylates/hydroxyesters acrylates copolymers of butyl acrylate, methyl methacrylate, methacrylic acid, ethyl acrylate and hydroxyethyl methacrylate; polyurethane-14/AMP-acrylates copolymer blend; and mixtures thereof. This includes both the acids and salts thereof.

### Other Ingredients

Other optional ingredients may be present in the aqueous spray compositions of the present invention. For example the aqueous spray compositions may further comprise: colourants/dyes, preservatives, viscosity control agents, microcapsules comprising benefit agents, structurants/dispersants, solvents, antifoams for processing aid etc.

### Spray Bottle

The compositions are fabric spray compositions. By this is meant that the compositions are suitable for spraying onto a fabric. They may be sprayed by any suitable spraying device.

Preferably the spray device is a manually operable spray device in the sense that the spray mechanism is manually operable to discharge a dose of said composition from the nozzle. The spray mechanism may be operated by an actuator. The actuator can be a push actuator or a pull actuator. The actuator may comprise a trigger. The spray mechanism may comprise a hand-operable pump. Optionally, said pump is one of: a positive displacement pump; a self-priming pump; a reciprocating pump. Suitable spray devices include trigger sprays, continuous/semi-continuous sprays, finger pump sprays, vibrating mesh device output sprays.

Preferably the spray device is operable without the use of a propellant. Indeed, propellant-free spray devices are preferred. This allows the spray to maintain the integrity and purity of the product, uncontaminated with propellant and is preferably environmentally.

Preferably the spray device is pressurised. This can improve spray duration and velocity. Preferably the spray device is pressurised by a gas chamber, separate from the reservoir containing the composition. The gas is preferably air or nitrogen. The spray device may comprise an outer container containing the composition and a pressurizing agent, wherein the composition is segregated from the pressurizing agent by containment (preferably hermetically sealed) in a flexible pouch. This which maintains complete formulation integrity so that only pure (i.e. excludes pressurising agent) composition is dispensed. Preferred systems are the so-called ‘bag-in-can’ (or BOV, bag-on-valve technology). Alternatively the spray device may comprise piston barrier mechanism, for example EarthSafe by Crown Holdings.

Preferably the spray device comprises a biodegradable plastic material.

The spray mechanism may further comprise an atomiser configured to break up said liquid dose into droplets and thereby facilitate creation of said fine aerosol in the form of a mist. Conveniently, said atomiser may comprise at least one of: a swirl chamber and a lateral dispersion chamber. Suitably, the atomiser functions to mix air with the aqueous fabric spray composition.

The particle size of the formulation when sprayed is preferably no more than 300 μm, preferably no more than 250 μm, preferably no more than 150 μm, preferably no more than 125 μm, preferably no more than 100 μm. The particle size of the formulation when sprayed is preferably at least 5 μm, preferably at least 10 μm, preferably at least 15 μm, preferably at least 20 μm, preferably at least 30 μm, preferably at least 40 μm. Suitably the spray comprises droplets having an average diameter in the range of preferably 5 to 300 μm, more preferably 10 to 250 μm, most preferably 15 to 150 μm.

This size allows for homogeneous distribution and a balance between sufficient wetting of the fabric, without potential fabric damage caused by excessive dosing of certain ingredients. Droplet size may be measured on a Malvern Spraytec instrument, with the peak maximum corresponding to the average droplet size. The parameter droplet size is the volume mean diameter, D[4,3].

Suitably, following actuation, the spray has a duration in the range of at least 0.4 seconds. Preferably the spray has a duration of at least 0.8 seconds. A longer duration minimises the effort by maximising coverage per actuation of a spray device. This is an important factor for products designed to be used over the full area of garments. Preferably the spray duration is directly linked to actuation such that the spray output continues only as long as the actuator is activated (e.g. as long as a button or trigger is pressed).

Spray reservoirs may be non-pressurised, manually or mechanically pre-pressurised devices. The above also to removable/refillable reservoirs.

According to a further aspect of the present invention, there is provided a replacement reservoir for a garment refresh product according to the above aspect(s), the replacement reservoir being pre-filled with a volume of said garment refreshing composition for replenishment of said product. A suitable “refill kit” comprises one or more reservoirs. In the case of more than one reservoir, for example two, three, four, five, or more reservoirs, the contents (aqueous fabric spray composition) of each reservoir may the same as or different from the other reservoirs.

### Dose

Conveniently, the garment refreshing composition is provided as a liquid, and said spray mechanism is operable to discharge a dose of at least 0.1 ml, preferably at least 0.2 ml, more preferably at least 0.25 ml, more preferably at least 0.3 ml, more preferably at least 0.35 ml, more preferably at least 0.35 ml, more preferably at least 0.4 ml, more preferably at least 0.45 ml, and most preferably at least 0.5 ml.

Suitably the dose is no more than 2 ml, preferably no more than 1.8 ml, preferably no more than 1.6 ml, more preferably no more than 1.5 ml, more preferably no more than 1.4 ml, more preferably no more than 1.3 ml, and most preferably no more than 1.2 ml.

Suitably the dose is between 0.1 and 2 ml of said liquid garment refreshing composition, preferably between 0.2 and 1.8 ml, more preferably 0.25 to 1.6 ml, more preferably 0.25 to 1.5 ml, and most preferably 0.25 to 1.2 ml.

These doses have been found to be particularly effective at achieving the desired garment refresh effect without unsightly and wasteful large droplet formation.

The dose may alternatively be defined as ml per m² of fabric. Preferably the spray composition of the present invention is dosed as 0.1 to 20 ml per m². More preferably 0.5 to 15 ml per m² and most preferably 1 to 10 ml per m².

### Method of Use

In one aspect of the present invention, there is provided a method of providing perfume to fabric. More particularly a method of providing an enduring perfume scent to fabric. i.e. the perfume scent lasts for longer.

### Use

In one aspect of the present invention, there is provided a use of the composition according to the present invention. The composition may be used to provide perfume to fabric. More particularly to provide an enduring perfume scent to fabric. i.e. the perfume scent lasts for longer.

### EXAMPLES

TABLE 2
Example formulations

| | Compositions (wt. %) | | | | | |
|---|---|---|---|---|---|---|
| | A | 1 | B | 2 | C | 3 |
| Perfume | 0.16 | 0.16 | — | — | — | — |
| composition A | | | | | | |
| Perfume | — | — | 0.3 | 0.3 | — | — |
| composition B | | | | | | |
| Perfume | — | — | — | — | 0.4 | 0.4 |
| composition C | | | | | | |
| Benzalkonium | — | 1 | — | 1 | — | 1 |
| chloride | | | | | | |
| PEG-40 | 0.080 | 0.08 | 0.08 | 0.08 | 1.5 | 1.5 |
| hydrogenated | | | | | | |
| castor oil | | | | | | |
| Malodour | 0.02 | 0.02 | 0.04 | 0.04 | 0.03 | 0.03 |
| counteractant | | | | | | |
| Amino silicone | 7 | 7 | 14 | 14 | — | — |
| emulsion¹ | | | | | | |
| Polyacrylate | 0.5 | 0.5 | — | — | — | — |
| co-polymer | | | | | | |
| Minors | <1 | <1 | <1 | <1 | <1 | <1 |
| Water | To | To | To | To | To | To |
| | 100 | 100 | 100 | 100 | 100 | 100 |

Amino silicone emulsion¹ - FC222 ex. Wacker Chemie
Perfume compositions A, B and C comprise at least 30 wt. % of the perfume composition, perfume ingredients having a LogP of 1 to 5 and a boiling point of greater than 150° C.

The non-ionic and perfume were pre-mixed at ˜55° C. The premix was then added to the water, at room temperature. The silicone and polyacrylate co-polymer were added to the compositions with mixing. The compositions were then discharged and tested.

### Assessment Method

The perfume benefit was analysed using VOC (volatile organic compound) headspace analysis with SPME-GC-MS.

1.0 g of product (from Table 2) was weighed accurately and decanted in to a 20 ml sampling vial. The sample vial was equilibrated for 10 minutes at 35° C. The sample vial was then loaded and run using a Shimadzu QP2010 plus GC/MS instrument, under the following conditions:
SPME fibre: divinylbenzene/carboxe/polydimethylsiloxane, sampling time 1 minute at 35° C.
GC oven: 45° C.-250° C. at 25° C./minute
Injection: 250° C. 40:1 split
GC Column: HP5 MS 20 m×0.18 mm×0.18 μm film thickness

Data was collected and expressed as summed GC/MS peak areas normalised for mass.

TABLE 3
Head space analysis results:

| | SPME-HS above product |
|---|---|
| A | 16689619 |
| 1 | 15634135 |
| B | 40835966 |
| 2 | 37806294 |
| C | 7697318 |
| 3 | 7045800 |

The head space above the products containing benzalkonium chloride have a lower head space count above the product. This demonstrates lower rates of evaporation, leading to more product being delivered to the fabric surface.

1. An aqueous fabric spray composition, comprising:
a. 0.0001-10 wt, % free perfume;
b. 0.001 to 2 wt. % quaternary ammonium biocide comprising benzyl C 12-16 alkyl dimethyl chloride; and
wherein at least 25 wt. % of the free perfume comprises perfume ingredient having a LogP of 1 to 5.

2. The aqueous fabric spray composition, according to claim 1, wherein the quaternary ammonium biocide: perfume are present in a weight ratio of 15:1 to 1:15.

3. The aqueous fabric spray composition, according to claim 1, wherein the composition further comprises non-ionic surfactant.

4. The aqueous fabric spray composition, according to claim 1, wherein the composition further comprises anti-malodour ingredient.

5. The aqueous fabric spray composition, according to claim 1, wherein the composition further comprises lubricant.

6. The aqueous fabric spray composition, according to claim 1, wherein the composition further comprises setting polymer.

7. The aqueous fabric spray composition, according to claim 1, wherein in the composition is contained in a spray bottle.

8. A method of providing perfume to fabric, wherein the spray compositions according to claim 1 is sprayed onto a fabric surface.

9. The method of providing perfume to fabric according to claim 8, wherein 0.1 to 20 ml per m² of the spray composition is sprayed onto the fabric surface.

10. The method of providing perfume to fabric according to claim 8 wherein the spray composition comprises from 0.005 to 1.5 wt. % biocide, from 0.001 to 8 wt. % free perfume having perfume ingredient with a LogP of 1 to 5, and anti-malodour ingredient comprising activated carbon, zeolite, metallic salt, cyclodextrin, reactive aldehyde, flavonoid or a mixture thereof.
